(19) United States
(12) Patent Application Publication
(10) Pub. No.: US 2020/0129156 A1
(43) Pub. Date: Apr. 30, 2020

(54) METHODS AND APPARATUS FOR COLLECTING COLOR DOPPLER ULTRASOUND DATA

(71) Applicants: David Elgena, Jersey City, NJ (US); Vineet Shah, Jersey City, NJ (US); Matthew de Jonge, Brooklyn, NY (US); Christophe Meyer, New York, NY (US)

(72) Inventors: David Elgena, Jersey City, NJ (US); Vineet Shah, Jersey City, NJ (US); Matthew de Jonge, Brooklyn, NY (US); Christophe Meyer, New York, NY (US)

(73) Assignee: Butterfly Network, Inc., Guilford, CT (US)

(21) Appl. No.: 16/663,120

(22) Filed: Oct. 24, 2019

Related U.S. Application Data
(60) Provisional application No. 62/750,385, filed on Oct. 25, 2018.

(51) Int. Cl. A61B 8/00; A61B 8/08; G06F 3/0484; G06F 3/0488; G06F 3/0482

(52) U.S. Cl. CPC A61B 8/465; A61B 8/488; G06F 3/0482; G06F 3/04883; G06F 3/04845

(57) ABSTRACT

Aspects of the technology described herein include a processing device configured to display, on a touch-sensitive display screen of a processing device in operative communication with an ultrasound device, an ultrasound image, a target region identifier superimposed on the ultrasound image, a first icon located on the target region identifier, and a second icon located on the target region identifier. The first icon is configured to control the height of the target region identifier and the angle of two opposite sides of the target region identifier. The second icon is configured to control the width of the target region identifier. The processing device is configured to configure the ultrasound device to collect color Doppler ultrasound data based on the region of the ultrasound image covered by the target region identifier and the angle of the two opposite sides of the target region identifier.

### CROSS-REFERENCE TO RELATED APPLICATIONS

This application claims the benefit under 35 U.S.C. § 119(e) of U.S. Patent Application Ser. No. 62/750,385, filed Oct. 25, 2018 under Attorney Docket No. B1348.70114US00, and entitled “METHODS AND APPARATUS FOR COLLECTING COLOR DOPPLER ULTRASOUND DATA”, which is hereby incorporated herein by reference in its entirety.

### FIELD

Generally, the aspects of the technology described herein relate to ultrasound data collection. Some aspects relate to collecting color Doppler ultrasound data.

### BACKGROUND

Ultrasound systems may be used to perform diagnostic imaging and/or treatment, using sound waves with frequencies that are higher with respect to those audible to humans.

Ultrasound imaging may be used to see internal soft tissue body structures, for example to find a source of disease or to exclude any pathology. When pulses of ultrasound are transmitted into tissue (e.g., by using a pulser in an ultrasound imaging device), sound waves are reflected off the tissue, with different tissues reflecting varying degrees of sound. These reflected sound waves may then be recorded and displayed as an ultrasound image to the operator. The strength (amplitude) of the sound signal and the time it takes for the wave to travel through the body provide information used to produce the ultrasound image. Many different types of images can be formed using ultrasound systems, including real-time images. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to one aspect, a method includes using two icons displayed by a processing device in operative communication with an ultrasound device to control three degrees of freedom of a target region identifier displayed by the processing device; and configuring the ultrasound device to collect color Doppler ultrasound data based on the target region identifier.

According to one aspect, a method includes using a first number of icons displayed by a processing device in operative communication with an ultrasound device to control a second number of degrees of freedom of a target region identifier displayed by the processing device, the second number being greater than the first number; and configuring the ultrasound device to collect color Doppler ultrasound data based on the target region identifier.

According to another aspect, a method includes displaying, on a touch-sensitive display screen of a processing device in operative communication with an ultrasound device: an ultrasound image, a target region identifier superimposed on the ultrasound image, a first icon located on the target region identifier, and a second icon located on the target region identifier, where the first icon is configured to control a height of the target region identifier and an angle of two opposite sides of the target region identifier; and the second icon is configured to control a width of the target region identifier; and configuring the ultrasound device to collect color Doppler ultrasound data based on the target region identifier.

In some embodiments, configuring the ultrasound device to collect the color Doppler ultrasound data based on the target region identifier comprises configuring the ultrasound device to collect the color Doppler ultrasound data based on a region of the ultrasound image covered by the target region identifier and the angle of the two opposite sides of the target region identifier. In some embodiments, the method further includes detecting a dragging movement covering a distance in a vertical direction across the touch-sensitive display screen, where the dragging movement begins on or within a threshold distance of the first icon; and changing a height of the target region identifier based on the distance in the vertical direction covered by the dragging movement. In some embodiments, the method further includes detecting a dragging movement covering a distance in a horizontal direction across the touch-sensitive display screen, where the dragging movement begins on or within the threshold distance of the first icon; and changing an angle of two opposite sides of the target region identifier based on the distance in the horizontal direction covered by the dragging movement. In some embodiments, the method further includes detecting a dragging movement covering a distance in a horizontal direction across the touch-sensitive display screen, where the dragging movement begins on or within the threshold distance of the second icon; and changing a width of the target region identifier based on the distance in the horizontal direction covered by the dragging movement.

According to another aspect, a method includes displaying, on a touch-sensitive display screen of a processing device in operative communication with an ultrasound device: an ultrasound image and a target region identifier superimposed on the ultrasound image; detecting a first dragging movement covering a distance in a vertical direction and/or a distance in a horizontal direction across the touch-sensitive display screen, where the dragging movement begins in an interior of the target region identifier, on the target region identifier, or outside but within a threshold distance of the target region identifier; changing a position of the target region identifier based on the distance in the horizontal direction and/or the distance in the vertical direction covered by the dragging movement; and configuring the ultrasound device to collect color Doppler ultrasound data based on the target region identifier.

In some embodiments, configuring the ultrasound device to collect the color Doppler ultrasound data based on the target region identifier comprises configuring the ultrasound device to collect the color Doppler ultrasound data based on a region of the ultrasound image covered by the target region identifier.

Some aspects include at least one non-transitory computer-readable storage medium storing processor-executable instructions that, when executed by at least one processor, cause the at least one processor to perform the above aspects and embodiments. Some aspects include an ultrasound system having a processing device configured to perform the above aspects and embodiments.

### BRIEF DESCRIPTION OF THE DRAWINGS

Various aspects and embodiments of the application will be described with reference to the following figures. It should be appreciated that the figures are not necessarily drawn to scale. Items appearing in multiple figures are indicated by the same reference number in all the figures in which they appear.

FIG. 1 illustrates an example graphical user interface GUI that is displayed on a touch-sensitive display screen of a processing device in an ultrasound system, in accordance with certain embodiments described herein.

FIG. 2 illustrates another example of the graphical user interface of FIG. 1, in accordance with certain embodiments described herein;

FIG. 3 illustrates another example of the graphical user interface of FIG. 1, in accordance with certain embodiments described herein;

FIG. 4 illustrates another example of the graphical user interface of FIG. 1, in accordance with certain embodiments described herein;

FIG. 5 illustrates another example of the graphical user interface of FIG. 1, in accordance with certain embodiments described herein;

FIG. 6 illustrates another example of the graphical user interface of FIG. 1, in accordance with certain embodiments described herein;

FIG. 7 illustrates an alternative example for the form of the box of FIGS. 1-6, in accordance with certain embodiments described herein;

FIG. 8 illustrates another alternative example for the form of the box of FIGS. 1-6, in accordance with certain embodiments described herein;

FIG. 9 illustrates another alternative example for the form of the box of FIGS. 1-6, in accordance with certain embodiments described herein:

FIG. 10 illustrates an example process for collecting color Doppler ultrasound data, in accordance with certain embodiments described herein;

FIG. 11 illustrates another example process for collecting color Doppler ultrasound data, in accordance with certain embodiments described herein;

FIG. 12 illustrates an example of another target region identifier that may be used to control collection of color Doppler ultrasound data, in accordance with certain embodiments described herein;

FIG. 13 illustrates another example of the target region identifier of FIG. 12, in accordance with certain embodiments described herein:

FIG. 14 illustrates another example of the target region identifier of FIG. 12, in accordance with certain embodiments described herein;

FIG. 15 illustrates a schematic block diagram illustrating aspects of an example ultrasound system upon which various aspects of the technology described herein may be practiced; and

FIG. 16 is a schematic block diagram illustrating aspects of another example ultrasound system upon which various aspects of the technology described herein may be practiced.

### DETAILED DESCRIPTION

Conventional ultrasound systems are large, complex, and expensive systems that are typically only purchased by large medical facilities with significant financial resources. Recently, cheaper and less complex ultrasound imaging devices have been introduced. Such imaging devices may include ultrasonic transducers monolithically integrated onto a single semiconductor die to form a monolithic ultrasound device. Aspects of such ultrasound-on-a chip devices are described in U.S. patent application Ser. No. 15/415,434 titled “UNIVERSAL ULTRASOUND DEVICE AND RELATED APPARATUS AND METHODS,” filed on Jan. 25, 2017 (and assigned to the assignee of the instant application) and published as U.S. Pat. Pub. No. US-2017-0360397-A1, which is incorporated by reference herein in its entirety. Such an ultrasound device may be in operative communication with a processing device, such as a smartphone or a tablet, having a touch-sensitive display screen. The processing device may display ultrasound images generated from ultrasound data collected by the ultrasound device.

The inventors have developed technology for assisting a user in controlling collection of color Doppler ultrasound data with an ultrasound device. Color Doppler ultrasound data may indicate the velocity of fluid flowing in a region exposed to ultrasound energy. The technology includes a target region identifier, such as a target region window or a box, that is displayed on the touch-sensitive display screen and superimposed on an ultrasound image depicted by the touch-sensitive display screen. The processing device may configure an ultrasound device to collect color Doppler ultrasound data based on the region of the ultrasound image covered by the box and the angle of two opposite sides of the box. A user may control collection of color Doppler data by modifying multiple parameters of the box, such as the position of the box, the height of the box, the width of the box, and the angle of the right and left sides of the box. To assist a user is modifying these parameters, the technology further includes two icons, a first icon and a second icon, that are displayed on the box. Based on detecting a dragging movement covering a distance in the vertical direction across the touch-sensitive display screen, where the dragging movement begins on or within a threshold distance of the first icon, the processing device may change the height of the box. Based on detecting a dragging movement covering a distance in the horizontal direction across the touch-sensitive display screen, where the dragging movement begins on or within a threshold distance of the first icon, the processing device may change the angle of the right and left sides of the box (where the angle of the right and left sides of the box is measured from the vertical direction of the touch-sensitive display screen). Based on detecting a dragging movement covering a distance in the horizontal direction across the touch-sensitive display screen, where the dragging movement begins on or within a threshold distance of the second icon, the processing device may change the width of the box. Additionally, based on detecting a dragging movement covering a distance in the horizontal direction and/or a distance in the vertical direction across the touch-sensitive display screen, where the dragging movement begins interior of the box, on the box, or outside but within a threshold distance of the box, the processing device may change the position of the box based on the distance in the horizontal direction and/or the distance in the vertical direction covered by the dragging movement. Generally, the technology may include using a certain number of regions of the touch-sensitive display screen to control more degrees of freedom of the box than the number of regions. For example, two icons on the box may control three degrees of freedom of the box: width, height, and angle of opposite sides of the box. This technology may provide a means of flexibly controlling collection of color Doppler ultrasound data that avoids excessively complicated selections of options on the touch-sensitive display screen and excessive crowding of the touch-sensitive display screen with controls.

It should be appreciated that the embodiments described herein may be implemented in any of numerous ways. Examples of specific implementations are provided below for illustrative purposes only. It should be appreciated that these embodiments and the features/capabilities provided may be used individually, all together, or in any combination of two or more, as aspects of the technology described herein are not limited in this respect.

While the description below includes certain methods that a processing device may use to cause a given result to occur, a processing device may implement different methods in order to cause the same result to occur. In particular, code designed to cause the result to occur may implement a different method to cause the result to occur than those described.

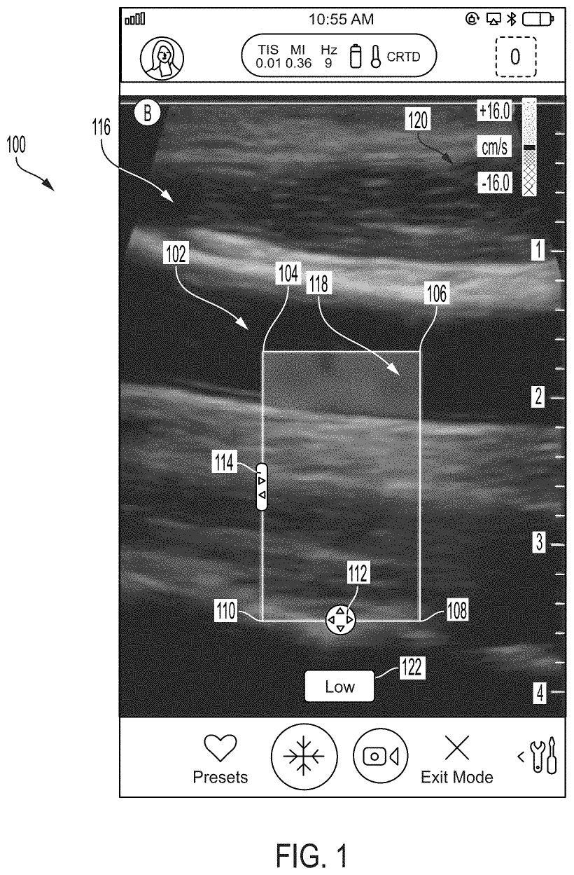

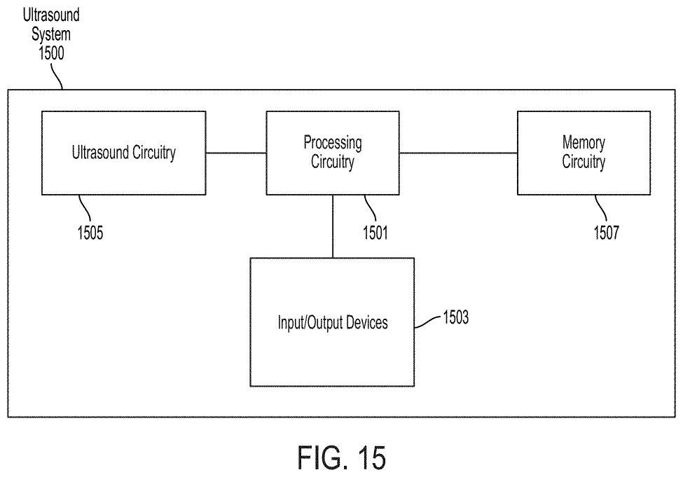

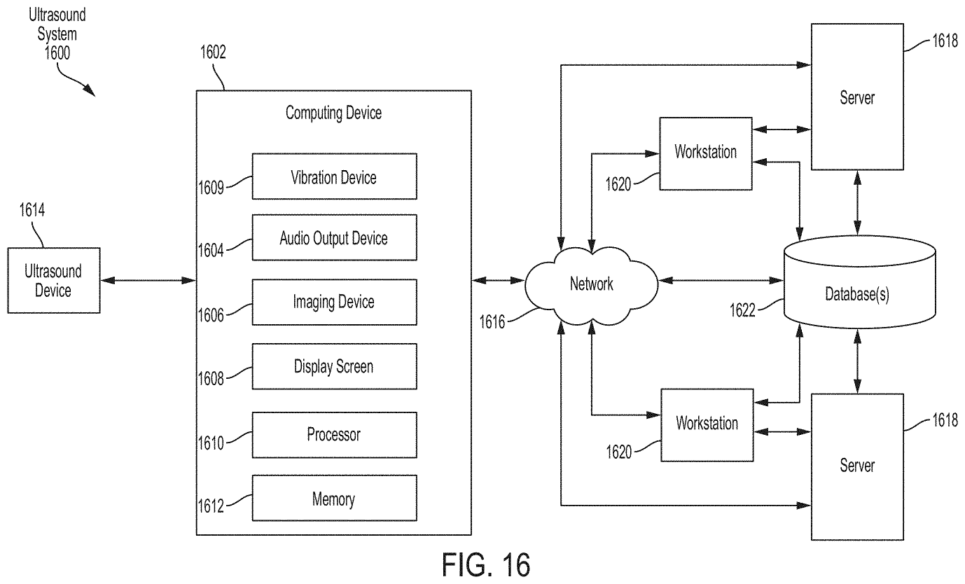

FIGS. 1-6 illustrate an example graphical user interface (GUI) 100 that is displayed on a touch-sensitive display screen of a processing device in an ultrasound system, in accordance with certain embodiments described herein. The GUI 100 is used for collecting color Doppler ultrasound data. The processing device is in operative communication with an ultrasound device (not shown in FIGS. 1-6). Ultrasound systems and devices are described in more detail with reference to FIGS. 15-16.

FIG. 1 illustrates an example of the GUI 100 that includes a box 102, a first icon 112, a second icon 114, a scale 120, a range control option 122, a brightness-mode (B-mode) ultrasound image 116, and a color Doppler ultrasound image 118. As referred to herein, a “box” need not necessarily be limited to a square or rectangle shape, but may also describe a trapezoid, parallelogram, or other polygon or closed region for example. More generally, aspects of the present application may use a target region identifier, of which a box is one non-limiting example. In some embodiments, the target region identifier may be a target region window. The following description refers primarily to a box for simplicity of description.

The color Doppler ultrasound image 118 is superimposed on the B-mode ultrasound image 116. The B-mode ultrasound image 116 may display data indicating the acoustic properties of a region exposed to ultrasound energy, while the color Doppler ultrasound image 118 may display data indicating the velocity of fluid flowing in the region. A specific portion of the color Doppler ultrasound image 118 that is superimposed on a specific portion of the B-mode ultrasound image 116 may indicate that the data in each portion was collected from the same spatial region. The scale 120 may indicate the correspondences between colors and velocities in the color Doppler ultrasound image 118. Scales utilizing features other than color, such as shading or patterning, may alternatively be implemented. In FIGS. 1-3, the top end of the scale, closer to +16.0 cm/s, is represented by the color blue, with the lower end of the scale, closer to −16.0 cm/s, is represented by the color red, with a steady gradation in color from one end to the other. As shown in those figures, the color blue is represented by a dot patter and the color red by a cross-hatching pattern. The ultrasound image 118 uses the same patterns.

The box 102 is superimposed on the B-mode ultrasound image 116. The box 102 includes a first vertex 104, a second vertex 106, a third vertex 108, and a fourth vertex 110. The first icon 112 is located on the box 102 approximately halfway between the third vertex 108 and the fourth vertex 110. The second icon 114 is located on the box 102 approximately halfway between the fourth vertex 110 and the first vertex 104. However, in some embodiments, the first icon 112 and/or the second icon 114 may be located on different portions of the respective sides of the box 102 (e.g., not necessarily approximately halfway). The first icon 112 includes four arrows pointing up, right, down, and left, which may indicate that dragging movements beginning on or within a threshold distance of the first icon 112 and proceeding in either the horizontal or vertical direction of the touch-sensitive display screen may cause a change to the box 102. The second icon 114 includes two arrows pointing left and right, which may indicate that dragging movements beginning on or within a threshold distance of the first icon 112 and proceeding in the horizontal direction of the touch-sensitive display screen may cause a change to the box 102. It should be appreciated, however, that the first icon 112 and the second icon 114 may have other forms.

As described above, each portion of the B-mode ultrasound image 116 and the color Doppler ultrasound image 118 displays data collected from a particular spatial region. Based on the particular spatial region from which data displayed by the B-mode ultrasound image 116 within the box 102 is collected, the processing device may configure the ultrasound device to focus ultrasound pulses on that same spatial region for producing color Doppler ultrasound data. In other words, the processing device may configure the ultrasound device to collect color Doppler ultrasound data by focusing ultrasound pulses on a spatial region corresponding to the region of the B-mode ultrasound image 116 that is covered by the box 102. To do this, the processing device may configure the ultrasound device to use a particular portion of the ultrasound device's transducer array for transmitting and receiving ultrasound pulses. The processing device may configure the ultrasound device such that transmit and receive lines cover the spatial region, but with a margin that may allow post-processing filters to correctly process data at the boundaries of the spatial region. The processing device may also configure beamforming circuitry in the ultrasound device to reconstruct scanlines focused on the particular spatial region. Color Doppler ultrasound data may then be shown by the color Doppler ultrasound image 118. The processing device may also exclude any color Doppler ultrasound data collected from spatial regions outside of the spatial region corresponding to the box 102 from being displayed by the color Doppler ultrasound image 118. If the box 102 is moved to a different location relative to the B-mode ultrasound image 116, the processing device may reconfigure the ultrasound device based on the new spatial region corresponding to the new region of the B-mode ultrasound image 116 covered by the box 102.

It should be appreciated that while it may appear in certain figures that color Doppler data is only displayed in a portion of the box 102, this is not due to color Doppler data not being collected in other portions of the box 102. Rather, the velocities in these other portions of the box 102 are sufficiently close to zero such that the color Doppler data appears non-existent.

The processing device may also configure the ultrasound device to collect color Doppler ultrasound data by tilting the transmitted ultrasound pulses based on the angle of the left and right sides of the box 102. For example, if the left and right sides of the box 102 are straight up and down (i.e., the angle is 0 degrees) on the touch-sensitive display screen, then the processing device may configure the ultrasound device to transmit ultrasound devices straight down. If the left and right sides of the box 102 are angled 45 degrees measured from the vertical axis of the touch-sensitive display screen, then the processing device may configure the ultrasound device to transmit ultrasound devices angled 45 degrees from straight down. Thus, the processing device may configure the ultrasound device to collect color Doppler ultrasound data based on the region of the B-mode ultrasound image 116 covered by the box 102 and based on the angle of the left and right sides of the box 102.

The position and shape of the box 102 relative to the B-mode ultrasound image 116 as shown in FIG. 1 may be a default position and shape when a user chooses color Doppler mode on the processing device. However, the default position and shape shown is not limiting, and other default positions and shape may be used. For example, there may be different default positions and shapes depending on the preset (e.g., the set of imaging parameters optimized for imaging a particular type of anatomy) that is selected.

The inventors have developed technology for assisting a user in modifying the region of the ultrasound image covered by the box 102 and the angle of the right and left sides of the box 102 using a touch-sensitive display screen. In some embodiments, the processing device may change the position of the box 102 based on a dragging movement that begins in the interior of the box 102, on the box 102, or outside but within a threshold distance of the box 102. A dragging movement may include, for example, a user touching his/her finger to the touch-sensitive display and dragging his/her finger to a different location on the touch-sensitive display screen. In particular, if a dragging movement that begins in the interior of the box 102, on the box 102, or outside but within a threshold distance of the box 102 covers a certain distance in the horizontal direction and/or a certain distance in the vertical direction, the processing device may change the location of every point on the box 102 by that same distance in the horizontal direction and/or distance in the vertical direction. (A dragging movement that covers a certain distance in a certain direction need not mean that the dragging movement actually proceeded along that direction, but rather that the dragging movement had a component along that direction. For example, a dragging movement in an arbitrary direction across a touch-sensitive display screen may have a component along the horizontal direction and a component along the vertical direction of the touch-sensitive display screen). Thus, the processing device may change the position of the box 102 based on the distance in the horizontal direction and/or the distance in the vertical direction covered by the dragging movement.

The touch-sensitive display screen may have an array of pixels, each pixel having a location that is x pixels in the horizontal direction and a location that is y pixels in the vertical location, where x and y are measured from an origin (e.g., a corner of the touch-sensitive display screen). As an example, when a user performs a dragging movement that begins in the interior of the box 102, on the box 102, or outside but within a threshold distance of the box 102 at a starting location (d1x, d1y) and ends at an ending location (d2x, d2y), the processing device may change the location of every point on the box 102 by a distance of (d2x−d1x, d2y−d1y). In this context, distance may be signed, where a negative distance may indicate a distance to the right of the touch-sensitive display screen and a positive distance may indicate a distance to the left of the touch-sensitive display screen, or vice versa, depending on the origin of the touch-sensitive display screen. In some embodiments, the processing device may change the locations of the first vertex 104, the second vertex 106, the third vertex 108, and the fourth vertex 110 by (d2x−d1x, d2y−d1y), and display the other points on the box between the new locations of the first vertex 104 and the second vertex 106, the second vertex 106 and the third vertex 108, the third vertex 108 and the fourth vertex 110, and the fourth vertex 110 and the first vertex 104, using the Cartesian equation for a line. The processing device may also change the location of the first icon 112 to be on the box 102 approximately halfway between the new location of the third vertex 108 and the fourth vertex 110, and change the location of the second icon 114 to be displayed on the box 102 approximately halfway between the new locations of the fourth vertex 110 and the first vertex 104. In some embodiments, the processing device may change the locations of the first icon 112 and the second icon 114 by a distance of (d2x−d1x, d2y−d1y).

Thus, more generally, it should be appreciated that the processing device may generate control signals to provide to the ultrasound device to control the ultrasound device to collect color Doppler data in a spatial region corresponding to a target region identifier displayed on the processing device. Furthermore, the processing device may receive input, for example user input, on the positioning, size, and orientation of the target region identifier. Additional examples are provided below.

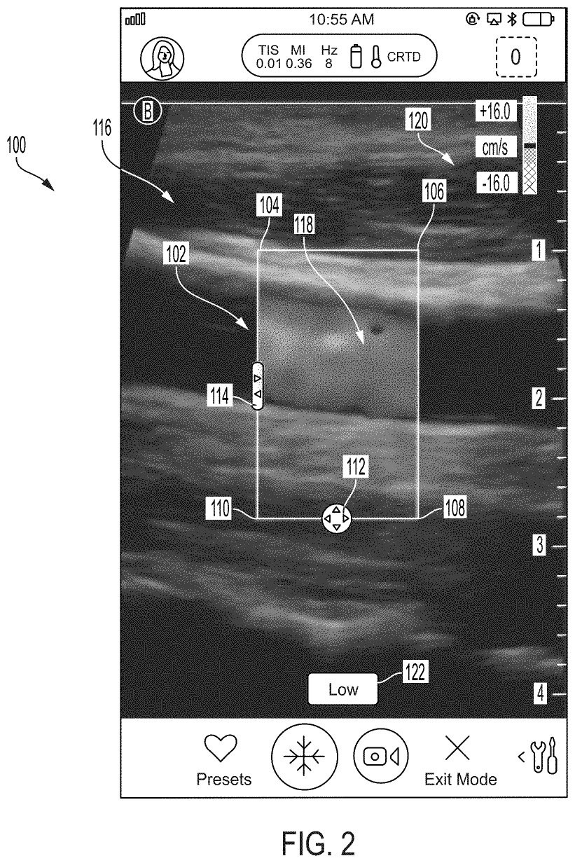

FIG. 2 illustrates another example of the GUI 100 after a dragging movement beginning in the interior of the box 102, on the box 102, or outside but within a threshold distance of the box 102. Prior to the dragging movement, the GUI 100 may have appeared as shown in FIG. 1. The processing device has changed the position of the box 102 by the distance in the horizontal direction and/or distance in the vertical direction covered by the dragging movement. The processing device has also changed the location of the first icon 112 to be on the box 102 approximately halfway between new locations of the third vertex 108 and the fourth vertex 110 and the location of the second icon 114 to be on the box 102 approximately halfway between the new locations of the fourth vertex 110 and the first vertex 104. The color Doppler ultrasound image 118 has also changed based on the new region of the B-mode ultrasound image 116 covered by the box 102. As described above, the processing device may configure the ultrasound device to collect color Doppler ultrasound data based on the region of the B-mode ultrasound image 116 that is covered by the box 102

The technology for assisting a user in modifying the region of the ultrasound image covered by the box 102 and the angle of the right and left sides of the box 102 using a touch-sensitive display screen also includes the first icon 112 and the second icon 114. The processing device may change the locations of the first vertex 104, the second vertex 106, the third vertex 108, and the fourth vertex 110 based on a dragging movement on the touch-sensitive display screen that begins on or within a threshold distance of the second icon 114. In particular, if a dragging movement that begins on or within a threshold distance of the second icon 114 covers a certain distance in the horizontal direction of the touch-sensitive display screen, the processing device may change the location of the first vertex 104 and the location of the fourth vertex 110 by that same distance in the horizontal direction, and the processing device may change the location of the second vertex 106 and the location of the third vertex 108 by the negative of that distance in the horizontal direction. For example, if the dragging movement covers a distance to the left of the touch-sensitive display screen, the processing device may change the location of the first vertex 104 and the location of the fourth vertex 110 by that same distance to the left of the touch-sensitive display screen, and change the location of the second vertex 106 and the location of the third vertex 108 by that same distance to the right of the touch-sensitive display screen. If the dragging movement covers a distance to the right of the touch-sensitive display screen, the processing device may change the location of the first vertex 104 and the location of the fourth vertex 110 by that same distance to the right of the touch-sensitive display screen, and change the location of the second vertex 106 and the location of the third vertex 108 by that same distance to the left of the touch-sensitive display screen. The processing device may thereby change the width of the box 102 based on the distance in the horizontal direction covered by the dragging movement.

For example, when a user performs a dragging movement that begins on or within a threshold distance of the second icon 114 at a starting location (d1x, d1y) and ends at an ending location (d2x, d2y), the processing device may change the locations of the first vertex 104 and the fourth vertex 110 by a distance of (d2x−d1x, 0) and change the locations of the second vertex 106 and the third vertex 108 by a distance of (−(d2x−d1x), 0). In some embodiments, the processing device may change the locations of every point on the box 102 between the first vertex 104 and the fourth vertex 110 by a distance of (d2x−d1x, 0) and change the locations of every point on the box 102 between the second vertex 106 and the third vertex 108 by a distance of (−(d2x−d1x), 0). In other embodiments, the processing device may determine the new locations of every point on the box 102 between the first vertex 104 and the fourth vertex 110 and the new locations of every point on the box 102 between the second vertex 106 and the third vertex 108 based on the Cartesian equation of a line. In some embodiments, the processing device may determine the new locations of every point on the box 102 between the first vertex 104 and the second 106 and the new locations of every point on the box 102 between the third vertex 108 and the fourth vertex 110 based on the Cartesian equation of a line. The processing device may also change the location of the second icon 114 to be on the box 102 approximately halfway between the new locations of the fourth vertex 110 and the first vertex 104. In some embodiments, the processing device may change the location of the second icon 114 by a distance of (d2x−d1x, 0).

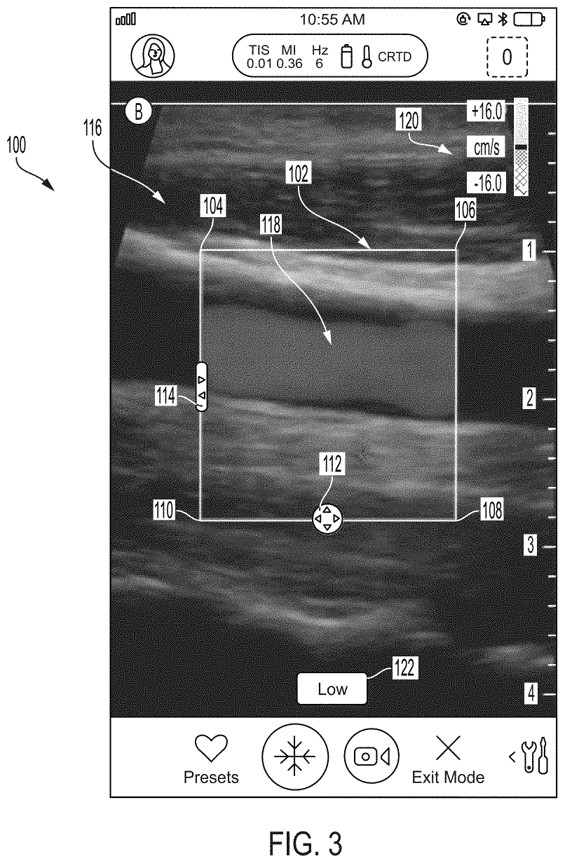

FIG. 3 illustrates another example of the GUI 100 after a dragging movement beginning on or within a threshold distance of the second icon 114. Prior to the dragging movement, the GUI 100 may have appeared as shown in FIG. 2. The processing device has changed the locations of the first vertex 104 and the fourth vertex 110 by the distance in the horizontal direction covered by the dragging movement and changed the locations of the second vertex 106 and the third vertex 108 by the negative distance in the horizontal direction covered by the dragging movement. The processing device has thereby changed the width of the box 102. The processing device has also changed the location of the second icon 114 to be on the box 102 approximately halfway between the new locations of the first vertex 104 and the fourth vertex 110. The color Doppler ultrasound image 118 has also changed based on the new region of the B-mode ultrasound image 116 covered by the box 102. As described above, the processing device may configure the ultrasound device to collect color Doppler ultrasound data based on the region of the B-mode ultrasound image 116 that is covered by the box 102.

The processing device may change the location of the fourth vertex 110 and the location of the third vertex 108 based on a dragging movement on the touch-sensitive display screen that begins on or within a threshold distance of the first icon 112. In particular, if a dragging movement that begins on or within a threshold distance of the first icon 112 covers a certain distance in the horizontal direction of the touch-sensitive display screen, the processing device may change the location of the fourth vertex 110 and the location of the third vertex 108 by that same distance in the horizontal direction. The processing device may thereby change the angle of the right and left sides of the box 102 based on the distance in the horizontal direction covered by the dragging movement.

For example, when a user performs a dragging movement that begins on or within a threshold distance of the first icon 112 at a starting location (d1x, d1y) and ends at an ending location (d2x, d2y), the processing device may change the location of the fourth vertex 110 and the third vertex 108 by a distance of (d2x−d1x, 0). In some embodiments, the processing device may also change the locations of every point on the box 102 between the fourth vertex 110 and the third vertex 108 by a distance of (d2x−d1x, 0). In other embodiments, the processing device may determine the locations for other points on the box 102 between the new locations of the fourth vertex 110 and the third vertex 108 based on the Cartesian equation of a line. In some embodiments, the processing device may determine the locations for other points on the box 102 between the first vertex 104 and the new location of the fourth vertex 110, and the second vertex 106 and the new location of the third vertex 108, based on the Cartesian equation of a line. The processing device may also change the location of the first icon 112 to be on the box 102 approximately halfway between the new locations of the third vertex 108 and the fourth vertex 110 and change the location of the second icon 114 to be displayed on the box 102 approximately halfway between the new locations of the fourth vertex 110 and the first vertex 104. In some embodiments, the processing device may change the locations of the first icon 112 and the second icon 114 by a distance of ((d2x−d1x)/2, 0). The second icon 114 may also be rotated by the angle by which the left side of the box 102 has been rotated.

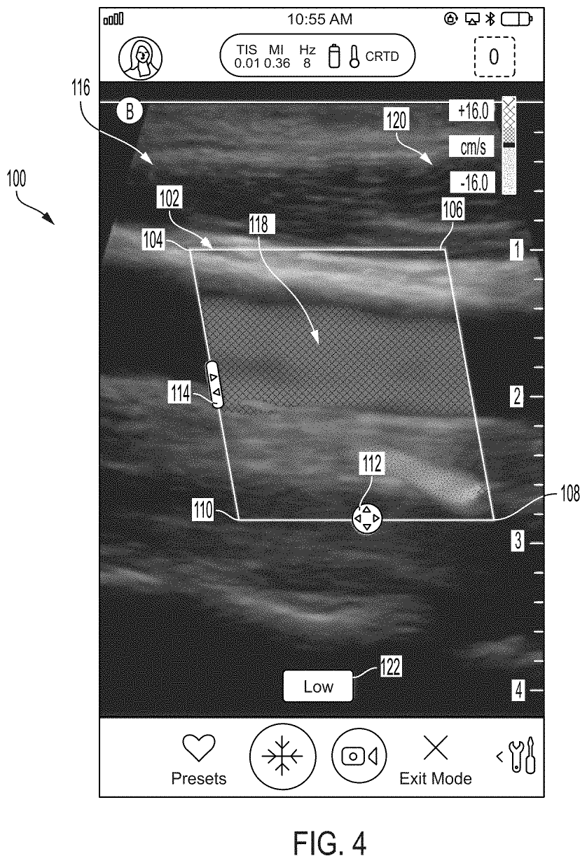

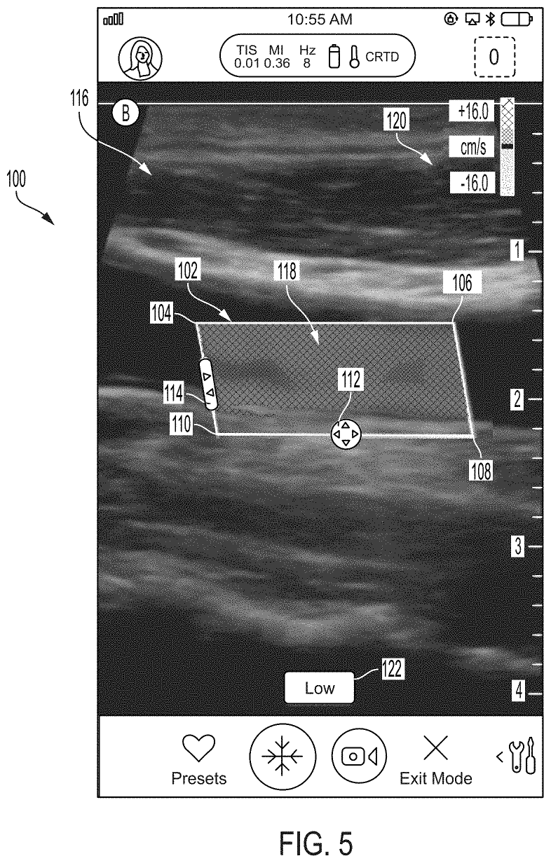

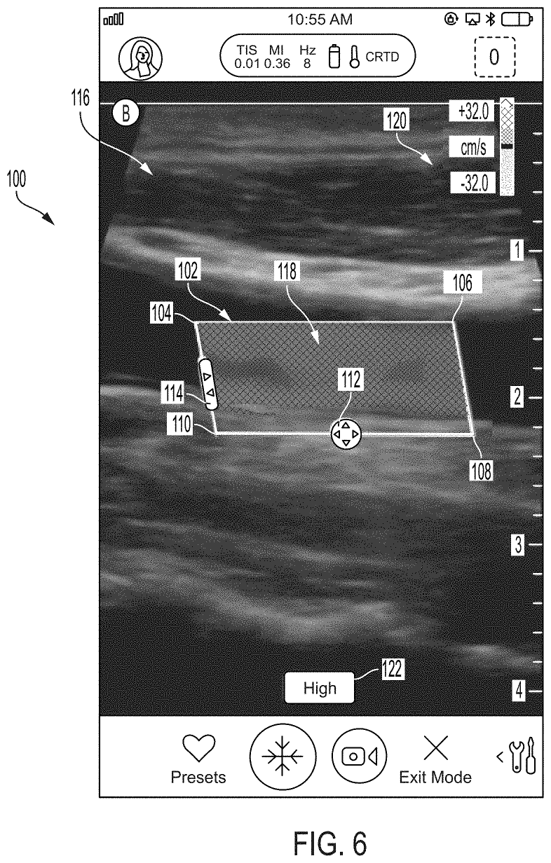

FIG. 4 illustrates another example of the GUI 100 after a dragging movement beginning on or within a threshold distance of the first icon 112. In FIGS. 4-6, the top end of the scale, closer to +16.0 cm/s in FIGS. 4-5 and closer to +32.0 cm/s in FIG. 6, is represented by the color red, with the lower end of the scale, closer to −16.0 cm/s in FIGS. 4-5 and −32.0 cm/s in FIG. 6, is represented by the color blue, with a steady gradation in color from one end to the other. As shown in those figures, the color blue is represented by a dot patter and the color red by a cross-hatching pattern. The ultrasound image 118 uses the same patterns. Referring to FIG. 4, prior to the dragging movement, the GUI 100 may have appeared as shown in FIG. 3. The processing device has changed the locations of the fourth vertex 110 and the third vertex 108 by the distance in the horizontal direction covered by the dragging movement. The processing device has thereby changed the angle of the left and right sides of the box 102. The processing device has also changed the location of the first icon 112 to be on the box 102 approximately halfway between the new locations of the fourth vertex 110 and the third vertex 108, and the location of the second icon 114 to be on the box 102 approximately halfway between the location of the first vertex 104 and the new location of the fourth vertex 110. The color Doppler ultrasound image 118 has also changed based on the new region of the B-mode ultrasound image 116 covered by the box 102 and by the modified angle of the left and right sides of the box 102. As described above, the processing device may configure the ultrasound device to collect color Doppler ultrasound data based on the region of the B-mode ultrasound image 116 that is covered by the box 102 and based on the angle of the left and right sides of the box 102.

The processing device may change the location of the first vertex 104, the second vertex 106, the third vertex 108, and the fourth vertex 110 based on a dragging movement on the touch-sensitive display screen that begins on or within a threshold distance of the first icon 112. In particular, if a dragging movement that begins on or within a threshold distance of the first icon 112 covers a certain distance in the vertical direction of the touch-sensitive display screen, the processing device may change the location of the third vertex 108 and the location of the fourth vertex 110 by that same distance in the vertical direction, and the processing device may change the location of the first vertex 104 and the second vertex 106 by the negative of that distance in the horizontal direction. For example, if the dragging movement covers a distance upwards on the touch-sensitive display screen, the processing device may change the location of the third vertex 108 and the location of the fourth vertex 110 by that same distance upwards on the touch-sensitive display screen, and change the location of the first vertex 104 and the location of the second vertex 106 by that same distance downwards on the touch-sensitive display screen. If the dragging movement covers a distance downwards on the touch-sensitive display screen, the processing device may change the location of the third vertex 108 and the location of the fourth vertex 110 by that same distance downwards on the touch-sensitive display screen, and change the location of the first vertex 104 and the location of the second vertex 106 by that same distance upwards on the touch-sensitive display screen. The processing device may thereby change the height of the box 102 based on the distance in the vertical direction covered by the dragging movement.

For example, when a user performs a dragging movement that begins on or within a threshold distance of the first icon 112 at a starting location (d1x, d1y) and ends at an ending location (d2x, d2y), the processing device may change the locations of the third vertex 108 and the fourth vertex 110 by a distance of (0, d2y−d1y) and change the locations of the first vertex 104 and the second vertex 106 by a distance of (0, −(d2y-d1y)). In some embodiments, the processing device may change the locations of every point on the box 102 between the third vertex 108 and the fourth vertex 110 by a distance of (0, d2y−d1y) and change the locations of every point on the box 102 between the first vertex 104 and the second vertex 106 by a distance of (0, −(d2y−d1y)). In other embodiments, the processing device may determine the new locations of every point on the box 102 between the first vertex 104 and the second vertex 106 and the new locations of every point on the box 102 between the third vertex 108 and the fourth vertex 110 based on the Cartesian equation of a line. In some embodiments, the processing device may determine the new locations of every point on the box 102 between the first vertex 104 and the fourth vertex 110 and the new locations of every point on the box 102 between the second vertex 106 and the third vertex 108 based on the Cartesian equation of a line. The processing device may also change the location of the first icon 112 to be on the box 102 approximately halfway between the new locations of the third vertex 108 and the fourth vertex 110. In some embodiments, the processing device may change the location of the first icon 112 by a distance of (0, d2y−d1y).

FIG. 5 illustrates another example of the GUI 100 after a dragging movement beginning on or within a threshold distance of the first icon 112. Prior to the dragging movement, the GUI 100 may have appeared as shown in FIG. 4. The processing device has changed the locations of the third vertex 108 and the fourth vertex 110 by the distance in the vertical direction covered by the dragging movement and changed the locations of the first vertex 104 and the second vertex 106 by the negative distance in the vertical direction covered by the dragging movement. The processing device has thereby changed the height of the box 102. The processing device has also changed the location of the first icon 112 to be on the box 102 approximately halfway between the new locations of the third vertex 108 and the fourth vertex 110. The color Doppler ultrasound image 118 has also changed based on the new location of the box 102. As described above, the processing device may configure the ultrasound device to collect color Doppler ultrasound data based on the region of the B-mode ultrasound image 116 that is covered by the box 102.

In some embodiments, the processing device may control the range of velocities that the ultrasound device is configured to collect through the color Doppler ultrasound data. In some embodiments, there may be two possible ranges. For example, the absolute values of the maximum and minimum velocities of one range may be greater than the absolute values of the maximum and minimum velocities of the other range. As a specific example, one range may be from −16 cm/s to 16 cm/s and the other range may be from −32 cm/s to 32 cm/s. The range having lower absolute values of the maximum and minimum velocities may be helpful for detecting and visualizing low speed flows, and the range having higher absolute values of the maximum and minimum velocities may be helpful for detecting and visualizing high speed flows. The range control option 122 may include text indicating the current range. For example, when the absolute values of the maximum and minimum velocities of one range are greater than the absolute values of the maximum and minimum velocities of the other range, the range control option 122 may either display “Low” or “High.” The range may be controlled by the range control option 122. Selecting the range control option 122 may switch from one range to the other range. In some embodiments, when the range control option 122 is selected, the processing device may configure the ultrasound device to modify the pulse repetition interval of transmitted ultrasound pulses. A shorter pulse repetition interval may be helpful for a range having higher absolute values of the maximum and minimum velocities. A longer pulse repetition interval may be helpful for a range having lower absolute values of the maximum and minimum velocities. In some embodiments, when the range control option 122 is selected, the processing device may change text displayed by the range control option 122 (e.g., from “Low” to “High” or from “High” to “Low”). In some embodiments, when the range control option 122 is selected, the correspondences between colors and velocities as displayed by the scale 120 may be modified to accommodate a different range of velocities.

In some embodiments, the processing device may recompute the absolute values of the maximum and minimum velocities that are displayed by the scale 120 based on the box 102. For example, moving the location of the box 102 further from the transducer array, and/or making the box 102 larger in size, may reduce the absolute values of the maximum and minimum velocities that the ultrasound device can detect, and the processing device may adjust the absolute values of the maximum and minimum velocities that are displayed by the scale 120 to match what the ultrasound device can detect. The processing device may thereby adjust the absolute values of the maximum and minimum velocities on the scale 120 even without selection of the range control option 122.

FIG. 6 illustrates another example of the GUI 100 after selection of the range control option 122. Prior to selection of the range control option 122, the GUI 100 may have appeared as shown in FIG. 5. The processing device has changed the text displayed by the range control option 122 from “Low” to “High.” The processing device has also changed the correspondences between colors and velocities as displayed by the scale 120. In particular, in FIG. 6, the scale 120 ranges from −32 cm/s to 32 cm/s rather than −16 cm/s to 16 cm/s while the range of colors can remain the same.

While FIGS. 1-6 illustrate an example with two ranges, in some embodiments there may be more than two ranges, or just one range. In the latter case, the range control option 122 may be absent. While in FIGS. 1-6, the two ranges are symmetrical about 0 cm/s, in some embodiments one or more ranges may not be symmetrical about 0 cm/s. While the two ranges in FIGS. 1-6 are from −32 cm/s to 32 cm/s and from −16 cm/s to 16 cm/s, in some embodiments other ranges may be used. In some embodiments, a different GUI (e.g., different than the GUI 100) may be used to change ranges. In this case, the range control option 122 may be absent from the GUI 100.

In some embodiments, the first icon 112 may be absent. In such embodiments, to change the height of the box 102, the user may initiate a dragging movement in the vertical direction at some location on the box 102 between the third vertex 108 and the fourth vertex 110. To change the angle of the left and right sides of the box 102, the user may initiate a dragging movement in the horizontal direction at some location on the box between the third vertex 108 and the fourth vertex 110. The user may also change the height of the box 102 or the slant of the left and right sides of the box 102 by initiating dragging movements between the first vertex 104 and the second vertex 106. In some embodiments, the second icon 114 may be absent. In such embodiments, to change the width of the box 102, the user may initiate a dragging movement in the horizontal direction at some location on the box 102 between the first vertex 104 and the fourth vertex. The user may also change the width of the box 102 initiating a dragging movement between the second vertex 106 and the third vertex 108.

The above description has described changing the height of the box 102 based on a distance in the vertical direction covered by a dragging movement that starts at the first icon 112. In some embodiments, the processing device may change the height of the box 102 based on taps. In particular, a user may tap the first icon 112 and then another location on the touch-sensitive display screen. The processing device may then change the height of the box 102 based on the distance in the vertical direction between the two tapped locations. The above description has described changing the angle of the left and right sides of the box 102 based on a distance in the horizontal direction covered by a dragging movement that starts at the first icon 112. In some embodiments, the processing device may change the angle of the left and right sides of the box 102 based on taps. In particular, a user may tap the first icon 112 and then another location on the touch-sensitive display screen. The processing device may then change the angle of the left and right sides of the box 102 based on the distance in the horizontal direction between the two tapped locations. The above description has described changing the width of the box 102 based on a distance in the horizontal direction covered by a dragging movement that starts at the second icon 114. In some embodiments, the processing device may change the width of the box 102 based on taps. In particular, a user may tap the second icon 114 and then another location on the touch-sensitive display screen. The processing device may then change the width of the box 102 based on the distance in the horizontal direction between the two tapped locations. In some embodiments, the user may need to tap twice on the selected locations.

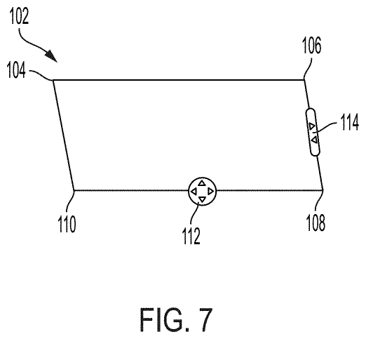

FIGS. 7-9 illustrate alternative examples for the form of the box 102, in accordance with certain embodiments described herein. For simplicity, only the box 102 is illustrated, without the rest of the GUI 100.

In FIG. 7, the box 102 differs from the box 102 of FIGS. 1-6 in that second icon 114 is located approximately halfway between the second vertex 106 and the third vertex 108. The processing device may change the locations of the first vertex 104, the second vertex 106, the third vertex 108, and the fourth vertex 110 based on a dragging movement on the touch-sensitive display screen that begins on or within a threshold distance of the second icon 114. In particular, if a dragging movement that begins on or within a threshold distance of the second icon 114 covers a certain distance in the horizontal direction of the touch-sensitive display screen, the processing device may change the location of the second vertex 106 and the location of the third vertex 108 by that same distance in the horizontal direction, and the processing device may change the location of the first vertex 104 and the location of the fourth vertex 110 by the negative of that distance in the horizontal direction. The processing device may also change the location of the second icon 114 to be on the box 102 approximately halfway between the new locations of the second vertex 106 and the third vertex 108. The processing device may thereby change the width of the box 102 based on the distance in the horizontal direction covered by the dragging movement. The behavior of the box 102 based on dragging movements that begin on or within a threshold distance of the first icon 112 may be same as described with reference to FIGS. 1-6 (particularly FIGS. 4-5).

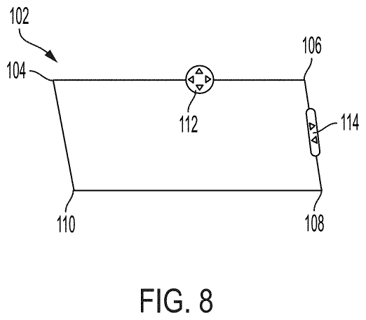

In FIG. 8, the box 102 differs from the box 102 of FIG. 7 in that the first icon 112 is located approximately halfway between the first vertex 104 and the second vertex 106. The processing device may change the location of the first vertex 104 and the location of the second vertex 106 based on a dragging movement on the touch-sensitive display screen that begins on or within a threshold distance of the first icon 112. In particular, if a dragging movement that begins on or within a threshold distance of the first icon 112 covers a certain distance in the horizontal direction of the touch-sensitive display screen, the processing device may change the location of the first vertex 104 and the location of the second vertex 106 by that same distance in the horizontal direction. The processing device may thereby change the angle of the right and left sides of the box 102 based on the distance in the horizontal direction covered by the dragging movement. The processing device may also change the location of the first icon 112 to be on the box 102 approximately halfway between the new locations of the first vertex 104 and the second vertex 106 and change the location of the second icon 114 to be displayed on the box 102 approximately halfway between the new locations of the second vertex 106 and the third vertex 108. The second icon 114 may also be rotated by the angle by which the right side of the box 102 has been rotated.

The processing device may also change the location of the first vertex 104, the second vertex 106, the third vertex 108, and the fourth vertex 110 based on a dragging movement on the touch-sensitive display screen that begins on or within a threshold distance of the first icon 112. In particular, if a dragging movement that begins on or within a threshold distance of the first icon 112 covers a certain distance in the vertical direction of the touch-sensitive display screen, the processing device may change the location of the first vertex 104 and the location of the second vertex 106 by that same distance in the vertical direction, and the processing device may change the location of the third vertex 108 and the fourth vertex 110 by the negative of that distance in the horizontal direction. The processing device may thereby change the height of the box 102 based on the distance in the vertical direction covered by the dragging movement. The processing device may also change the location of the first icon 112 to be on the box 102 approximately halfway between the new locations of the first vertex 104 and the second vertex 106. The behavior of the box 102 based on dragging movements that begin on or within a threshold distance of the second icon 114 may be same as described with reference to FIG. 7.

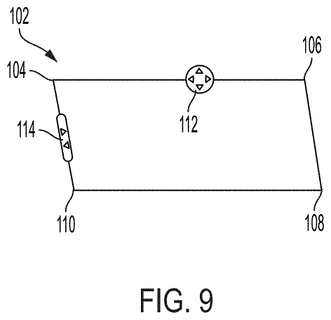

In FIG. 9, the box 102 differs from the box 102 of FIGS. 1-6 in that the first icon 112 is located approximately halfway between the first vertex 104 and the second vertex 106. The behavior of the box 102 based on dragging movements that begin on or within a threshold distance of the first icon 112 may be same as described with reference to FIG. 8. The behavior of the box 102 based on dragging movements that begin on or within a threshold distance of the first icon 112 may be same as described with reference to FIGS. 1-6 (particularly FIG. 3).

The processing device has been described as changing the angle of the left and right sides of the box 102. However, in some embodiments, the processing device may change the angle of the top and bottom sides of the box 102. For example, the processing device may change the locations of the first vertex 104 and the fourth vertex 110 or the locations of the second vertex 106 and the third vertex 108. In such embodiments, the first icon 112 may be displayed on the box 102 between the first vertex 104 and the fourth vertex 110 or between the second vertex 106 and the third vertex 108. Dragging movements that begin on or within a threshold distance of the first icon 112 may change the width of the box 102 and/or the angle of the top and bottom sides of the box 102. Additionally, in such embodiments, the second icon 114 may be displayed on the box 102 between the first vertex 104 and the second vertex 106 or between the third vertex 108 and the fourth vertex 110. Dragging movements that begin on or within a threshold distance of the second icon 114 may change the height of the box 102 and/or the angle of the top and bottom sides of the box 102. The second icon 114 may be displayed in such embodiments rotated 90 degrees from the orientation shown in FIG. 1 when the top and bottom sides of the box 102 are not angled.

The processing device has been described as changing the height or width of the box 102 by changing the locations of the first vertex 104, the second vertex 106, the third vertex 108, and the fourth vertex 110. However, in some embodiments, the processing device may change the height of the box 102 by only changing the location of the first vertex 104 and the second vertex 106 or by only changing the location of the third vertex 108 and the fourth vertex 110. In some embodiments, the processing device may change the width of the box 102 by only changing the location of the first vertex 104 and the fourth vertex 110 or by only changing the location of the second vertex 106 and the third vertex 108.

In some embodiments, the processing device may change the Doppler gain based on a dragging movement that begins outside the box 102 and not within a threshold distance of the first icon 112, the second icon 114, or the box 102 itself.

It should be understood that in some embodiments, certain portions of the GUI 100 (including features not described herein such as buttons and indicators) may be absent. Additionally, certain elements of the GUI 100, such as the range control option 122 and the scale 120 may be displayed in different locations than shown in the figures. While the above description has described that a processing device may perform certain calculations using pixels, in some embodiments the processing device may perform calculations using points. It should be noted that certain calculations described herein may produce fractional pixel results. In some embodiments, fractional pixel results may be rounded to a whole pixel. In some embodiments, the processing device may use antialiasing to interpret pixel values for a fractional pixel result (e.g., to interpret pixel values for pixels (1, 1) and (2, 1) when a calculation indicates that something should be displayed at pixel (1.5, 1)). As described above, the processing device may change the height of the box 102, the width of the box 102, and/or the angle of the right and left sides of the box 102 based on a dragging movement that begins on or within a threshold distance of the first icon 112, the second icon 114, or the box 102 itself. In some embodiments, the threshold distance may be measured in pixels (e.g., 30 pixels). While the above description has described a touch-sensitive display screen, in some embodiments the screen may not be a touch-sensitive display screen, and a click and dragging movement of a cursor (e.g., using a mouse) may be the equivalent of a dragging movement.

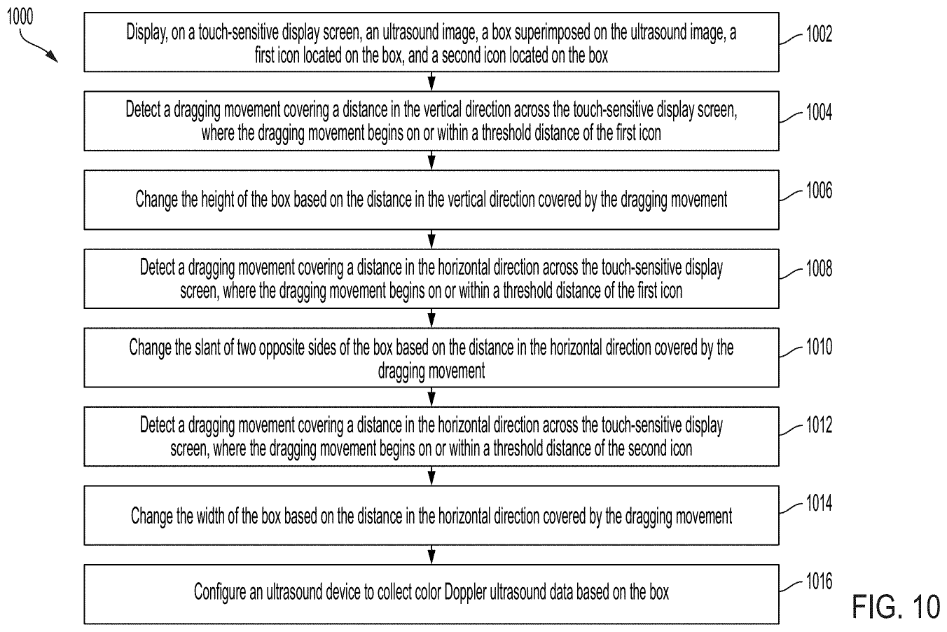

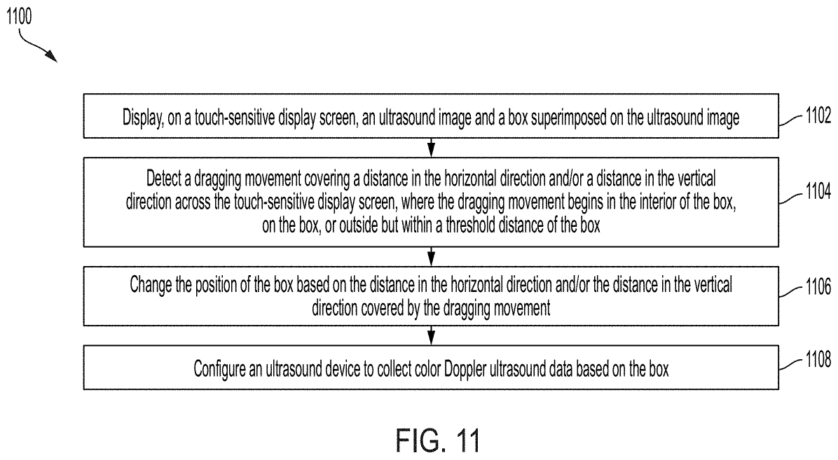

FIGS. 10-11 illustrate example processes for collecting color Doppler ultrasound data, in accordance with certain embodiments described herein. The processes may be performed by a processing device in an ultrasound system. The processing device may be, for example, a mobile phone, tablet, or laptop in operative communication with an ultrasound probe. The ultrasound probe and the processing device may communicate over a wired communication link (e.g., over Ethernet, a Universal Serial Bus (USB) cable or a Lightning cable) or over a wireless communication link (e.g., over a BLUETOOTH, WiFi, or ZIGBEE wireless communication link). Further description of the processes 1000 and 1100 may be found with reference to FIGS. 1-9.

FIG. 10 illustrates an example process 1000 for collecting color Doppler ultrasound data, in accordance with certain embodiments described herein.

In act 1002, the processing device displays, on a touch-sensitive display screen, (1) an ultrasound image (e.g., the B-mode ultrasound image 116), (2) a box (e.g., the box 102) superimposed on the ultrasound image, (3) a first icon (e.g., the first icon 112) located on the box, and (4) a second icon (e.g., the second icon 114) located on the box. The first icon may be configured to control the height of the box and the angle of two opposite sides of the box. The second icon may be configured to control the width of the box. The process 1000 proceeds from act 1002 to act 1004.

In act 1004, the processing device detects a dragging movement covering a distance in the vertical direction across the touch-sensitive display screen, where the dragging movement begins on or within a threshold distance of the first icon. The process 1000 proceeds from act 1004 to act 1006.

In act 1006, the processing device changes the height of the box based on the distance in the vertical direction covered by the dragging movement. The process 1000 proceeds from act 1006 to act 1008.

In act 1008, the processing device detects a dragging movement covering a distance in the horizontal direction across the touch-sensitive display screen, where the dragging movement begins on or within a threshold distance of the first icon. The process 1000 proceeds from act 1008 to act 1010.

In act 1010, the processing device changes the angle of two opposite sides of the box (e.g., the left and right sides of the box) based on the distance in the horizontal direction covered by the dragging movement. The process 1010 proceeds from act 1010 to act 1012.

In act 1012, the processing device detects a dragging movement covering a distance in the horizontal direction across the touch-sensitive display screen, where the dragging movement begins on or within a threshold distance of the second icon. The process 1000 proceeds from act 1012 to act 1014.

In act 1014, the processing device changes the width of the box based on the distance in the horizontal direction covered by the dragging movement. The process 1000 proceeds from act 1014 to act 1016.

In act 1016, the processing device configures the ultrasound device to collect color Doppler ultrasound data based on the box. In some embodiments, the processing device may configure the ultrasound device to collect color Doppler ultrasound data based on the region of the ultrasound image covered by the box and the angle of the two opposite sides of the box.

It should be appreciated that in some embodiments, certain acts of the process 1000 may be optional (e.g., acts 1004, 1006, 1008, 1010, 1012, or 1014).

FIG. 11 illustrates an example process 1100 for collecting color Doppler ultrasound data, in accordance with certain embodiments described herein.

In act 1102, the processing device displays, on a touch-sensitive display screen. (1) an ultrasound image (e.g., the B-mode ultrasound image 116), and (2) a box (e.g., the box 102) superimposed on the ultrasound image. The process 1100 proceeds from act 1102 to act 1104.

In act 1104, the processing device detects a dragging movement covering a distance in the horizontal direction and/or a distance in the vertical direction across the touch-sensitive display screen, where the dragging movement begins interior of the box, on the box, or outside but within a threshold distance of the box. The process 1100 proceeds from act 1104 to act 1106.

In act 1106, the processing device changes the position of the box based on the distance in the horizontal direction and/or the distance in the vertical direction covered by the dragging movement. The process 1100 proceeds from act 1106 to act 1108.

In act 1108, the processing device configures the ultrasound device to collect color Doppler ultrasound data based on the box. In some embodiments, the processing device may configure the ultrasound device to collect color Doppler ultrasound data based on the region of the ultrasound image covered by the box.

It should be appreciated that in some embodiments, certain acts of the process 1100 may be optional (e.g., acts 1104 or 1106).

Various inventive concepts may be embodied as one or more processes, of which examples have been provided. The acts performed as part of each process may be ordered in any suitable way. Thus, embodiments may be constructed in which acts are performed in an order different than illustrated, which may include performing some acts simultaneously, even though shown as sequential acts in illustrative embodiments. Further, one or more of the processes may be combined and/or omitted, and one or more of the processes may include additional steps.

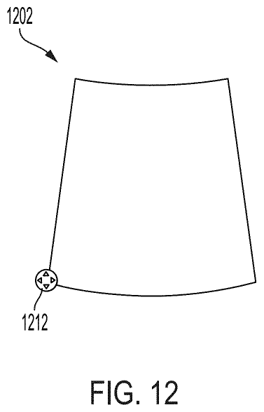

FIG. 12 illustrates an example of another target region identifier 1202 that may be used to control collection of color Doppler ultrasound data, in accordance with certain embodiments described herein. For simplicity, only the target region identifier 1202 is illustrated, without the rest of the GUI. In FIG. 12, the target region identifier 1202 is wedge- or sector-shaped. The target region identifier 1202 includes an icon 1212. The processing device may configure the ultrasound device to collect color Doppler ultrasound data based on the target region identifier 1202. In particular, based on the particular spatial region from which data displayed by the B-mode ultrasound image (not shown in figure) within the target region identifier 1202 is collected, the processing device may configure the ultrasound device to focus ultrasound pulses on that same spatial region for producing color Doppler ultrasound data. Additionally, the left and right sides of the target region identifier 1202 may control the virtual apex used for color Doppler ultrasound data collection. In particular, the right and left sides of the target region identifier 1202 may point to the virtual apex. The portion of an ultrasound device's ultrasound transducer array used to generate transmitted ultrasound pulses at any instantaneous time may be referred to as the instantaneous transmit aperture. The ultrasound device may transmit multiple ultrasound beams in multiple spatial directions in order to collect ultrasound data for forming a full ultrasound image. For each transmitted ultrasound beam using a particular instantaneous transmit aperture, one can consider a line extending from the center of the instantaneous transmit aperture along the direction of the transmitted ultrasound beam. The point in space where all such lines intersect for a given group of transmitted ultrasound beams used to form an ultrasound image may be referred to as the virtual apex. Thus, changing the angle of the right and left sides of the target region identifier 1202 may be used to control the virtual apex for data collection.

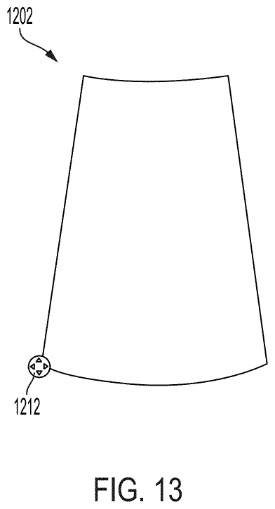

FIG. 13 illustrates another example of the target region identifier 1202, in accordance with certain embodiments described herein. FIG. 13 may illustrate the target region identifier 1202 after a dragging movement that begins on or within a threshold distance of the icon 1212 when the target region identifier 1202 is in the configuration of FIG. 12 and covers a distance in the vertical direction across the touch-sensitive display screen. The processing device may change the height of the target region identifier 1202 based on the distance in the vertical direction covered by the dragging movement. As seen in FIG. 13, the height of the target region identifier 1202 has changed from FIG. 12.

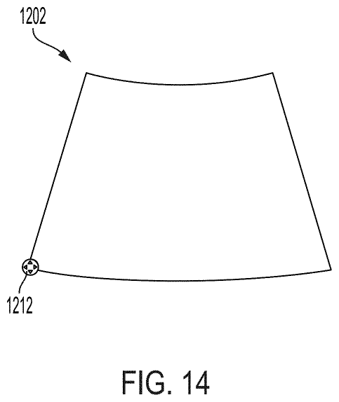

FIG. 14 illustrates another example of the target region identifier 1202, in accordance with certain embodiments described herein. FIG. 14 may illustrate the target region identifier 1202 after a dragging movement that begins on or within a threshold distance of the icon 1212 when the target region identifier 1202 is in the configuration of FIG. 13 and covers a distance in the horizontal direction across the touch-sensitive display screen. The processing device may change the width of the target region identifier 1202 based on the distance in the horizontal direction covered by the dragging movement. As seen in FIG. 14, the width of the target region identifier 1202 has changed from FIG. 13.

FIG. 15 illustrates a schematic block diagram illustrating aspects of an example ultrasound system 1500 upon which various aspects of the technology described herein may be practiced. For example, one or more components of the ultrasound system 1500 may perform any of the processes described herein. As shown, the ultrasound system 1500 includes processing circuitry 1501, input/output devices 1503, ultrasound circuitry 1505, and memory circuitry 1507.

The ultrasound circuitry 1505 may be configured to generate ultrasound data that may be employed to generate an ultrasound image. The ultrasound circuitry 1505 may include one or more ultrasonic transducers monolithically integrated onto a single semiconductor die. The ultrasonic transducers may include, for example, one or more capacitive micromachined ultrasonic transducers (CMUTs), one or more CMOS ultrasonic transducers (CUTs), one or more piezoelectric micromachined ultrasonic transducers (PMUTs), and/or one or more other suitable ultrasonic transducer cells. In some embodiments, the ultrasonic transducers may be formed the same chip as other electronic components in the ultrasound circuitry 1505 (e.g., transmit circuitry, receive circuitry, control circuitry, power management circuitry, and processing circuitry) to form a monolithic ultrasound imaging device.

The processing circuitry 1501 may be configured to perform any of the functionality described herein. The processing circuitry 1501 may include one or more processors (e.g., computer hardware processors). To perform one or more functions, the processing circuitry 1501 may execute one or more processor-executable instructions stored in the memory circuitry 1507. The memory circuitry 1507 may be used for storing programs and data during operation of the ultrasound system 1500. The memory circuitry 1507 may include one or more storage devices such as non-transitory computer-readable storage media. The processing circuitry 1501 may control writing data to and reading data from the memory circuitry 1507 in any suitable manner.

In some embodiments, the processing circuitry 1501 may include specially-programmed and/or special-purpose hardware such as an application-specific integrated circuit (ASIC). For example, the processing circuitry 1501 may include one or more graphics processing units (GPUs) and/or one or more tensor processing units (TPUs). TPUs may be ASICs specifically designed for machine learning (e.g., deep learning). The TPUs may be employed to, for example, accelerate the inference phase of a neural network.

The input/output (I/O) devices 1503 may be configured to facilitate communication with other systems and/or an operator. Example I/O devices 1503 that may facilitate communication with an operator include: a keyboard, a mouse, a trackball, a microphone, a touch-sensitive display screen, a printing device, a display screen, a speaker, and a vibration device. Example I/O devices 1503 that may facilitate communication with other systems include wired and/or wireless communication circuitry such as BLUETOOTH, ZIGBEE, Ethernet. WiFi. and/or USB communication circuitry.

It should be appreciated that the ultrasound system 1500 may be implemented using any number of devices. For example, the components of the ultrasound system 1500 may be integrated into a single device. In another example, the ultrasound circuitry 1505 may be integrated into an ultrasound imaging device that is communicatively coupled with a processing device that includes the processing circuitry 1501, the input/output devices 1503, and the memory circuitry 1507.

FIG. 16 is a schematic block diagram illustrating aspects of another example ultrasound system 1600 upon which various aspects of the technology described herein may be practiced. For example, one or more components of the ultrasound system 1600 may perform any of the processes described herein. As shown, the ultrasound system 1600 includes an ultrasound imaging device 1614 in wired and/or wireless communication with a processing device 1602. The processing device 1602 includes an audio output device 1604, an imaging device 1606, a display screen 1608, a processor 1610, a memory 1612, and a vibration device 1609. The processing device 1602 may communicate with one or more external devices over a network 1616. For example, the processing device 1602 may communicate with one or more workstations 1620, servers 1618, and/or databases 1622.

The ultrasound imaging device 1614 may be configured to generate ultrasound data that may be employed to generate an ultrasound image. The ultrasound imaging device 1614 may be constructed in any of a variety of ways. In some embodiments, the ultrasound imaging device 1614 includes a transmitter that transmits a signal to a transmit beamformer which in turn drives transducer elements within a transducer array to emit pulsed ultrasonic signals into a structure, such as a patient. The pulsed ultrasonic signals may be back-scattered from structures in the body, such as blood cells or muscular tissue, to produce echoes that return to the transducer elements. These echoes may then be converted into electrical signals by the transducer elements and the electrical signals are received by a receiver. The electrical signals representing the received echoes are sent to a receive beamformer that outputs ultrasound data.

The processing device 1602 may be configured to process the ultrasound data from the ultrasound imaging device 1614 to generate ultrasound images for display on the display screen 1608. The processing may be performed by, for example, the processor 1610. The processor 1610 may also be adapted to control the acquisition of ultrasound data with the ultrasound imaging device 1614. The ultrasound data may be processed in real-time during a scanning session as the echo signals are received. In some embodiments, the displayed ultrasound image may be updated a rate of at least 5 Hz, at least 10 Hz, at least 20 Hz, at a rate between 5 and 60 Hz, at a rate of more than 20 Hz. For example, ultrasound data may be acquired even as images are being generated based on previously acquired data and while a live ultrasound image is being displayed. As additional ultrasound data is acquired, additional frames or images generated from more-recently acquired ultrasound data are sequentially displayed. Additionally. or alternatively, the ultrasound data may be stored temporarily in a buffer during a scanning session and processed in less than real-time.

Additionally (or alternatively), the processing device 1602 may be configured to perform any of the processes described herein (e.g., using the processor 1610). For example, the processing device 1602 may be configured to automatically determine an anatomical feature being imaged and automatically select, based on the anatomical feature being imaged, an ultrasound imaging preset corresponding to the anatomical feature. As shown, the processing device 1602 may include one or more elements that may be used during the performance of such processes. For example, the processing device 1602 may include one or more processors 1610 (e.g., computer hardware processors) and one or more articles of manufacture that include non-transitory computer-readable storage media such as the memory 1612. The processor 1610 may control writing data to and reading data from the memory 1612 in any suitable manner. To perform any of the functionality described herein, the processor 1610 may execute one or more processor-executable instructions stored in one or more non-transitory computer-readable storage media (e.g., the memory 1612), which may serve as non-transitory computer-readable storage media storing processor-executable instructions for execution by the processor 1610.

In some embodiments, the processing device 1602 may include one or more input and/or output devices such as the audio output device 1604, the imaging device 1606, the display screen 1608, and the vibration device 1609. The audio output device 1604 may be a device that is configured to emit audible sound such as a speaker. The imaging device 1606 may be configured to detect light (e.g., visible light) to form an image such as a camera. The display screen 1608 may be configured to display images and/or videos such as a liquid crystal display (LCD), a plasma display, and/or an organic light emitting diode (OLED) display. The display screen 1618 may be a touch-sensitive display screen. The vibration device 1609 may be configured to vibrate one or more components of the processing device 1602 to provide tactile feedback. These input and/or output devices may be communicatively coupled to the processor 1610 and/or under the control of the processor 1610. The processor 1610 may control these devices in accordance with a process being executed by the processor 1610 (such as the processes shown in FIGS. 10-11). Similarly, the processor 1610 may control the audio output device 1604 to issue audible instructions and/or control the vibration device 1609 to change an intensity of tactile feedback (e.g., vibration) to issue tactile instructions. Additionally (or alternatively), the processor 1610 may control the imaging device 1606 to capture non-acoustic images of the ultrasound imaging device 1614 being used on a subject to provide an operator of the ultrasound imaging device 1614 an augmented reality interface.

It should be appreciated that the processing device 1602 may be implemented in any of a variety of ways. For example, the processing device 1602 may be implemented as a handheld device such as a mobile smartphone or a tablet. Thereby, an operator of the ultrasound imaging device 1614 may be able to operate the ultrasound imaging device 1614 with one hand and hold the processing device 1602 with another hand. In other examples, the processing device 1602 may be implemented as a portable device that is not a handheld device such as a laptop. In yet other examples, the processing device 1602 may be implemented as a stationary device such as a desktop computer.

In some embodiments, the processing device 1602 may communicate with one or more external devices via the network 1616. The processing device 1602 may be connected to the network 1616 over a wired connection (e.g., via an Ethernet cable) and/or a wireless connection (e.g., over a WiFi network). As shown in FIG. 16, these external devices may include servers 1618, workstations 1620, and/or databases 1622. The processing device 1602 may communicate with these devices to, for example, off-load computationally intensive tasks. For example, the processing device 1602 may send an ultrasound image over the network 1616 to the server 1618 for analysis (e.g., to identify an anatomical feature in the ultrasound) and receive the results of the analysis from the server 1618. Additionally (or alternatively), the processing device 1602 may communicate with these devices to access information that is not available locally and/or update a central information repository. For example, the processing device 1602 may access the medical records of a subject being imaged with the ultrasound imaging device 1614 from a file stored in the database 1622. In this example, the processing device 1602 may also provide one or more captured ultrasound images of the subject to the database 1622 to add to the medical record of the subject. For further description of ultrasound imaging devices and systems, see U.S. Patent Application Publication No. US20170360397A1 titled “UNIVERSAL ULTRASOUND DEVICE AND RELATED APPARATUS AND METHODS.”

Some aspects include at least one non-transitory computer-readable storage medium storing processor-executable instructions that, when executed by at least one processor, cause the at least one processor to perform the aspects and embodiments described herein. In some embodiments, at least one non-transitory computer-readable storage medium stores processor-executable instructions that, when executed by at least one processor, cause the at least one processor to display, on a touch-sensitive display screen of a processing device in operative communication with an ultrasound device, an ultrasound image, a target region identifier superimposed on the ultrasound image, a first icon located on the target region identifier, and a second icon located on the target region identifier. The first icon is configured to control a height of the target region identifier and an angle of two opposite sides of the target region identifier, and the second icon is configured to control a width of the target region identifier. The processor-executable instructions, when executed by the at least one processor, further cause the at least one processor to configure the ultrasound device to collect color Doppler ultrasound data based on a region of the ultrasound image covered by the target region identifier and the angle of the two opposite sides of the target region identifier. According to one aspect, the processor-executable instructions for configuring the ultrasound device to collect the color Doppler ultrasound data based on the target region identifier comprise processor-executable instructions for configuring the ultrasound device to collect the color Doppler ultrasound data based on a region of the ultrasound image covered by the target region identifier and the angle of the two opposite sides of the target region identifier. According to another aspect, the at least one non-transitory computer-readable storage medium further stores processor-executable instructions that, when executed by the at least one processor, cause the at least one processor to detect a dragging movement covering a distance in a vertical direction across the touch-sensitive display screen, wherein the dragging movement begins on or within a threshold distance of the first icon; and change a height of the target region identifier based on the distance in the vertical direction covered by the dragging movement.

In some embodiments, at least one non-transitory computer-readable storage medium stores processor-executable instructions that, when executed by at least one processor, cause the at least one processor to: display, on a touch sensitive display screen of a processing device in operative communication with an ultrasound device, an ultrasound image, and a target region identifier superimposed on the ultrasound image; detect a first dragging movement covering a distance in a vertical direction and/or a distance in a horizontal direction across the touch-sensitive display screen, wherein the dragging movement begins in an interior of the target region identifier, on the target region identifier, or outside but within a threshold distance of the target region identifier, change a position of the target region identifier based on the distance in the horizontal direction and/or the distance in the vertical direction covered by the dragging movement; and configure the ultrasound device to collect color Doppler ultrasound data based on the target region identifier. According to one aspect, the processor-executable instructions for configuring the ultrasound device to collect the color Doppler ultrasound data based on the target region identifier comprise processor-executable instructions for configuring the ultrasound device to collect the color Doppler ultrasound data based on a region of the ultrasound image covered by the target region identifier.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including.” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. An apparatus, comprising:
a processing device in operative communication with an ultrasound device, the processing device configured to:
display on a touch-sensitive display screen of the processing device:
an ultrasound image;
a target region identifier superimposed on the ultrasound image;
a first icon located on the target region identifier; and
a second icon located on the target region identifier;
use the first and second icons to control three degrees of freedom of the target region identifier; and
configure the ultrasound device to collect color Doppler ultrasound data based on the target region identifier.

2. The apparatus of claim 1, wherein the processing device is configured, when using the first and second icons to control the three degrees of freedom of the target region identifier, to:
use the first icon to control a height of the target region identifier and an angle of two opposite sides of the target region identifier; and
use the second icon to control a width of the target region identifier.

3. The apparatus of claim 2, wherein the processing device is configured, when configuring the ultrasound device to collect the color Doppler ultrasound data based on the target region identifier, to configure the ultrasound device to collect the color Doppler ultrasound data based on a region of the ultrasound image covered by the target region identifier and the angle of the two opposite sides of the target region identifier.

4. The apparatus of claim 2, wherein the processing device is configured, when using the first icon to control the height of the target region identifier and the angle of the two opposite sides of the target region identifier, to:
detect a dragging movement covering a distance in a vertical direction across the touch-sensitive display screen, wherein the dragging movement begins on or within a threshold distance of the first icon; and
change the height of the target region identifier based on the distance in the vertical direction covered by the dragging movement.

5. The apparatus of claim 2, wherein the processing device is configured, when using the first icon to control the height of the target region identifier and the angle of the two opposite sides of the target region identifier, to:
detect a dragging movement covering a distance in a horizontal direction across the touch-sensitive display screen, wherein the dragging movement begins on or within the threshold distance of the first icon; and
change the angle of the two opposite sides of the target region identifier based on the distance in the horizontal direction covered by the dragging movement.

6. The apparatus of claim 2, wherein the processing device is configured, when using the second icon to control the width of the target region identifier, to:
detect a dragging movement covering a distance in a horizontal direction across the touch-sensitive display screen, wherein the dragging movement begins on or within the threshold distance of the second icon; and
change the width of the target region identifier based on the distance in the horizontal direction covered by the dragging movement.

7. The apparatus of claim 1, wherein the processing device is further configured to:
detect a first dragging movement covering a distance in a vertical direction and/or a distance in a horizontal direction across the touch-sensitive display screen, wherein the dragging movement begins in an interior of the target region identifier, on the target region identifier, or outside but within a threshold distance of the target region identifier, and
change a position of the target region identifier based on the distance in the horizontal direction and/or the distance in the vertical direction covered by the dragging movement.

8. The apparatus of claim 7, wherein the processing device is configured, when configuring the ultrasound device to collect the color Doppler ultrasound data based on the target region identifier, to configure the ultrasound device to collect the color Doppler ultrasound data based on a region of the ultrasound image covered by the target region identifier.

9. The apparatus of claim 1, wherein the target region identifier is overlaid on the ultrasound image.

10. The apparatus of claim 1, wherein the three degrees of freedom comprise a height of the target region identifier, an angle of two opposite sides of the target region identifier, and a width of the target region identifier.

11. A method, comprising:
displaying on a touch-sensitive display screen of a processing device in operative communication with an ultrasound device:
an ultrasound image;
a target region identifier superimposed on the ultrasound image;
a first icon located on the target region identifier; and
a second icon located on the target region identifier,
using the first and second icons to control three degrees of freedom of the target region identifier; and
configuring the ultrasound device to collect color Doppler ultrasound data based on the target region identifier.

12. The method of claim 11, wherein using the first and second icons to control the three degrees of freedom of the target region identifier comprises:
using the first icon to control a height of the target region identifier and an angle of two opposite sides of the target region identifier, and
using the second icon to control a width of the target region identifier.

13. The method of claim 12, wherein configuring the ultrasound device to collect the color Doppler ultrasound data based on the target region identifier comprises configuring the ultrasound device to collect the color Doppler ultrasound data based on a region of the ultrasound image covered by the target region identifier and the angle of the two opposite sides of the target region identifier.

14. The method of claim 12, wherein using the first icon to control the height of the target region identifier and the angle of the two opposite sides of the target region identifier comprises:
detecting a dragging movement covering a distance in a vertical direction across the touch-sensitive display screen, wherein the dragging movement begins on or within a threshold distance of the first icon; and
changing the height of the target region identifier based on the distance in the vertical direction covered by the dragging movement.

15. The method of claim 12, wherein using the first icon to control the height of the target region identifier and the angle of the two opposite sides of the target region identifier comprises:
detecting a dragging movement covering a distance in a horizontal direction across the touch-sensitive display screen, wherein the dragging movement begins on or within the threshold distance of the first icon; and
changing the angle of the two opposite sides of the target region identifier based on the distance in the horizontal direction covered by the dragging movement.

16. The method of claim 12, wherein using the second icon to control the width of the target region identifier comprises:
detecting a dragging movement covering a distance in a horizontal direction across the touch-sensitive display screen, wherein the dragging movement begins on or within the threshold distance of the second icon; and
changing the width of the target region identifier based on the distance in the horizontal direction covered by the dragging movement.

17. The method of claim 11, further comprising:
detecting a first dragging movement covering a distance in a vertical direction and/or a distance in a horizontal direction across the touch-sensitive display screen, wherein the dragging movement begins in an interior of the target region identifier, on the target region identifier, or outside but within a threshold distance of the target region identifier, and
changing a position of the target region identifier based on the distance in the horizontal direction and/or the distance in the vertical direction covered by the dragging movement.

18. The method of claim 17, wherein configuring the ultrasound device to collect the color Doppler ultrasound data based on the target region identifier comprises configuring the ultrasound device to collect the color Doppler ultrasound data based on a region of the ultrasound image covered by the target region identifier.

19. The method of claim 11, wherein the target region identifier is overlaid on the ultrasound image.

20. The method of claim 11, wherein the three degrees of freedom comprise a height of the target region identifier, an angle of two opposite sides of the target region identifier, and a width of the target region identifier.
